(19) United States
(12) Patent Application Publication
(10) Pub. No.: US 2026/0325830 A1
(43) Pub. Date: Oct. 22, 2026

(54) THERAPEUTIC DEVICE AND ELECTRODE HEAD THEREFOR

(71) Applicant: AN GROUP LIMITED, HONG KONG (CN)

(72) Inventor: JUNYUAN CHEN, HONG KONG (CN)

(21) Appl. No.: 19/212,739

(22) Filed: May 20, 2025

(30) Foreign Application Priority Data
Apr. 17, 2025 (CN) 202520737412.7

(51) Int. Cl. A61N 1/04; A61N 1/36

(52) U.S. Cl. CPC A61N 1/0456; A61N 1/048; A61N 1/36021; A61N 1/36034

(57) ABSTRACT

A therapeutic device includes at least one wire, a main unit and at least one electrode connector. The at least one electrode connector includes an insulating enclosure and a conductive portion, the conductive portion including a contact surface; wherein a magnet is disposed within the conductive portion, the magnet being magnetized along an axial direction of the magnet; and the insulating enclosure includes a first end surface and a second end surface that are opposite along the axial direction of the magnet, the contact surface being exposed at the first end surface, the first region of the second end surface and the contact surface being both planar, the first region being magnetically attachable to the contact surface of another electrode connector, and a magnetic pole mounting direction of the magnet in each of the at least one electrode connector being consistent.

### CROSS-REFERENCE TO RELATED APPLICATIONS

This application claims priority to Chinese Patent Application No. 202520737412.7, filed with the Chinese Patent Office on Apr. 17, 2025, titled “THERAPEUTIC DEVICE AND ELECTRODE HEAD THEREFOR”, the entire contents of which are incorporated herein by reference.

### TECHNICAL FIELD

Embodiments of the present disclosure relate to the technical field of therapeutic devices, and in particular, relate to a therapeutic device and an electrode lead therefor.

### BACKGROUND

A therapeutic device stimulates nerve fibers to activate a pain suppression mechanism of a human body by applying low-frequency or medium-frequency electrical pulses to human skin. This mechanism helps alleviate or block transmission of pain signals to the brain, such that a therapeutic effect in pain relief is achieved. The therapeutic device is primarily constituted by a main unit and electrode pads. The electrode pads are attached to painful areas of the human body and are connected to the main unit via electrode leads. Electrical pulse stimulation signals output by the main unit are transmitted to the human body via the electrode pads.

Since the main unit of the therapeutic device requires the use of at least two electrode pads, it is necessary to equip the device with a plurality of electrode leads to establish connections between the main unit and the electrode pads. In a case where the number of electrode leads is large, the wires are prone to entangling with each other, which may damage the wires and cause the wires to become disorganized and untidy. This results in inconvenience for a user when organizing the wires, leading to a poor user experience.

### SUMMARY

In view of the above, embodiments of the present disclosure provide a therapeutic device and an electrode lead therefor for ease of storage.

Some embodiments of the present disclosure further provide an electrode lead for a therapeutic device. The electrode lead includes:
at least one wire;
a main unit connector disposed at a first end of the at least one wire, and electrically connected to a main unit of the therapeutic device; and
at least one electrode connector disposed at a second end of the at least one wire, wherein the at least one electrode connector includes an insulating enclosure and a conductive portion, the conductive portion being electrically connected to the main unit connector via the at least one wire, the conductive portion including a contact surface configured to mate with an electrode sheet, and the insulating enclosure covering the conductive portion with the contact surface exposed;
wherein a magnet is disposed within the conductive portion, the magnet being magnetized along an axial direction of the magnet, the axial direction of the magnet being perpendicular to the contact surface; and the insulating enclosure includes a first end surface and a second end surface that are opposite along the axial direction of the magnet, the contact surface being exposed at the first end surface, the first region of the second end surface and the contact surface being both planar, the first region being magnetically attachable to the contact surface of another electrode connector, and a magnetic pole mounting direction of the magnet in each of the at least one electrode connector being consistent.

In some embodiments, a protrusion is arranged on the second end surface of the insulating enclosure, wherein a top surface of the protrusion forms the first region; and a recess is defined in the first end surface of the insulating enclosure, wherein a shape and size of the recess are configured to be adapted for engagement with a protrusion of another electrode connector.

In some embodiments, a cross-section of the protrusion and a cross-section of the recess are both circular, or square, or triangular, or elliptical.

In some embodiments, a bottom surface of the recess is flush with the contact surface, and a height of the protrusion is equal to a depth of the recess.

In some embodiments, the insulating enclosure includes a first housing and a second housing, wherein a first engagement portion is arranged on the first housing, and a second engagement portion is arranged on the second housing, wherein the first engagement portion and the second engagement portion are engaged with each other to secure the first housing and the second housing.

In some embodiments, the first engagement portion is an arcuate recess, and the second engagement portion is an arcuate protrusion, wherein the arcuate recess is adapted to the arcuate protrusion in terms of shape and size.

In some embodiments, a through hole is defined in the first housing, wherein the through hole is configured to accommodate the conductive portion.

In some embodiments, the magnet is a ring-shaped body.

In some embodiments, a number of the at least one wire is n, and a number of the at least one electrode connector is also n, wherein n is a positive integer greater than 1, and the n electrode connectors are electrically connected to the main body connector via the n wires; and
the electrode lead for the therapeutic device is configured such that: in a non-use state, the n electrode connectors are disposed adjacently, and in any adjacent two of the n electrode connectors, a contact surface of one electrode connector is magnetically attached to a first region of the other electrode connector such that the electrode lead for the therapeutic device is stored.

In some embodiments, the at least one wire includes a first wire and a second wire, and the at least one electrode connector includes a first electrode connector and a second electrode connector, wherein conductive portions of the first and second electrode connectors are electrically connected to the main unit connector via the first and second wires respectively; and
the electrode lead for the therapeutic device is configured such that: in a non-use state, a contact surface of the first electrode connector is magnetically attached to a first region of the second electrode connector such that the electrode lead for the therapeutic device is stored.

In some embodiments, the at least one wire includes a first wire, a second wire, a third wire, and a fourth wire, and the at least one electrode connector includes a first electrode connector, a second electrode connector, a third electrode connector, and a fourth electrode connector, wherein conductive portions of the first, second, third, and fourth electrode connectors are electrically connected to the main unit connector via the first, second, third, and fourth wires respectively; and
the electrode lead for the therapeutic device is configured such that: a contact surface of the first electrode connector is magnetically attached to a first region of the second electrode connector, and a contact surface of the third electrode connector is magnetically attached to a first region of the fourth electrode connector, such that a pair of electrode connectors for single-channel use is obtained; and
the electrode lead for the therapeutic device is further configured such that: in a non-use state, the contact surface of the first electrode connector is magnetically attached to the first region of the second electrode connector, a contact surface of the second electrode connector is magnetically attached to a first region of the third electrode connector, and the contact surface of the third electrode connector is magnetically attached to the first region of the fourth electrode connector, such that the electrode lead for the therapeutic device is stored.

Some embodiments of the present disclosure further provide a therapeutic device. The therapeutic device includes:
the electrode head as described above; and
a main unit.

In some embodiments, a number of the at least one wire is n, and a number of the at least one electrode connector is n, wherein n is a positive integer greater than 1; and the main unit includes a controller, n switch units, and n current detection units, wherein the n switch units are in one-to-one correspondence with the n electrode connectors, the n current detection units are in one-to-one correspondence with the n electrode connectors, the n electrode connectors include a first electrode connector, the n switch units include a first switch unit in correspondence with the first electrode connector, and the n current detection units include a first current detection unit in correspondence with the first electrode connector, wherein the first switch unit is connected to a power terminal of the main unit, and the other switches in the n switch units are grounded;
wherein the controller is configured to control the first switch unit to be turned on and control a target switch unit in the other switch units to be turned on, and detect a current at the first electrode connector via the first current detection unit or detect a current at a target electrode connector corresponding to a target current detection unit in the n current detection units via the target current detection unit to determine whether a path is formed between the first electrode connector and the target electrode connector, wherein the target electrode connector is in correspondence with the target switch unit.

In some embodiments, the at least one wire includes a first wire, a second wire, and a third wire; the at least one electrode lead includes a first electrode lead, a second electrode lead, and a third electrode lead; and the main unit includes a controller, a first switch unit, a fourth switch unit, a sixth switch unit, a first current detection unit, a second current detection unit, and a third current detection unit; wherein the first switch unit, the fourth switch unit, and the sixth switch unit are in one-to-one correspondence with the first electrode lead, the second electrode lead, and the third electrode lead; the first current detection unit, the second current detection unit, and the third current detection unit are in one-to-one correspondence with the first electrode lead, the second electrode lead, and the third electrode lead; the first switch unit is connected to a power terminal of the main unit; and the fourth switch unit and the fourth switch unit are grounded;
wherein the controller is configured to control the first switch unit to be turned on and control a target switch unit in the fourth switch unit and the sixth switch unit to be turned on, and detect a current at the first electrode connector via the first current detection unit or detect a current at a target electrode connector corresponding to a target current detection unit in the second current detection unit and the third current detection unit via the target current detection unit to determine whether a path is formed between the first electrode connector and the target electrode connector, wherein the target electrode connector is in correspondence with the target switch unit.

In some embodiments, the at least one wire includes a first wire, a second wire, a third wire, and a fourth wire; the at least one electrode lead includes a first electrode lead, a second electrode lead, a third electrode lead, and a fourth electrode lead; and the main unit includes a controller, a first switch unit, a fourth switch unit, a sixth switch unit, an eighth switch unit, a first current detection unit, a second current detection unit, a third current detection unit, and a fourth current detection unit; wherein the first switch unit, the fourth switch unit, the sixth switch unit, and the eighth switch unit are in one-to-one correspondence with the first electrode lead, the second electrode lead, the third electrode lead, and the fourth electrode lead; the first current detection unit, the second current detection unit, the third current detection unit, and the fourth current detection are in one-to-one correspondence with the first electrode lead, the second electrode lead, the third electrode lead, and the fourth electrode lead; the first switch unit is connected to a power terminal of the main unit; and the fourth switch unit, the fourth switch unit, and the eighth switch unit are grounded;
wherein the controller is configured to control the first switch unit to be turned on and control a target switch unit in the fourth switch unit, the sixth switch unit, and the eight switch unit to be turned on, and detect a current at the first electrode connector via the first current detection unit or detect a current at a target electrode connector corresponding to a target current detection unit in the second current detection unit, the third current detection unit, and the fourth current detection unit via the target current detection unit to determine whether a path is formed between the first electrode connector and the target electrode connector, wherein the target electrode connector is in correspondence with the target switch unit.

In some embodiments, the at least one wire includes a first wire, a second wire, a third wire, and a fourth wire; the at least one electrode lead includes a first electrode lead, a second electrode lead, a third electrode lead, and a fourth electrode lead; and the main unit includes a controller, a first switch, a third switch, a fourth switch, a fifth switch unit, a sixth switch,, an eighth switch, a first current detection unit, a second current detection unit, a third current detection unit, and a fourth current detection unit; wherein the first electrode connector is connected to a power terminal of the main unit via the first switch, the second electrode is connected to the power terminal of the main unit via the third switch and grounded via the fourth switch, the third electrode connector is connected to the power terminal of the main unit via the fifth switch and grounded via the sixth switch, the fourth electrode connector is grounded via the eighth switch, and the first current detection unit, the second current detection unit, the third current detection unit, and the fourth current detection unit are respectively connected to the first electrode connector, the second electrode connector, the third electrode connector, and the fourth electrode connector.
the controller is configured to:
control the first switch and the fourth switch to be turned on, and detect a current at the first electrode connector via the first current detection unit or detect a current at the second electrode connector via the second current detection circuit to determine whether a path is formed between the first electrode connector and the second electrode connector;
control the first switch and the sixth switch to be turned on, and detect a current at the first electrode connector via the first current detection unit or detect a current at the third electrode connector via the third current detection circuit to determine whether a path is formed between the first electrode connector and the third electrode connector;
control the first switch and the eighth switch to be turned on, and detect a current at the first electrode connector via the first current detection unit or detect a current at the fourth electrode connector via the fourth current detection circuit to determine whether a path is formed between the first electrode connector and the fourth electrode connector;
control the third switch and the sixth switch to be turned on, and detect a current at the second electrode connector via the second current detection unit or detect a current at the third electrode connector via the third current detection circuit to determine whether a path is formed between the second electrode connector and the third electrode connector;
control the third switch and the eighth switch to be turned on, and detect a current at the second electrode connector via the second current detection unit or detect a current at the fourth electrode connector via the fourth current detection circuit to determine whether a path is formed between the second electrode connector and the fourth electrode connector; or
control the fifth switch and the eighth switch to be turned on, and detect a current at the third electrode connector via the third current detection unit or detect a current at the fourth electrode connector via the fourth current detection circuit to determine whether a path is formed between the third electrode connector and the fourth electrode connector.

In some embodiments, a first current control unit is further connected between the first switch and the power terminal of the main unit; and in a case where the first electrode connector forms a first path, a second path, or a third path with the second electrode connector, the third electrode connector, or the fourth electrode connector, the controller is configured to control a magnitude of current of the first path, the second path, or the third path via the first current control unit;
a second current control unit is further connected between the third switch and the power terminal of the main unit; and in a case where the first electrode connector forms a fourth path or a fifth path with the third electrode connector or the fourth electrode connector, the controller is configured to control a magnitude of current of the fourth path or the fifth path via the second current control unit; and
a third current control unit is further connected between the fourth switch and the power terminal of the main unit; and in a case where the third electrode connector forms a sixth path with the fourth electrode connector, the controller is configured to control a magnitude of current of the sixth path via the third current control unit.

In some embodiments, each of the first switch, the third switch, the fourth switch, the fifth switch, the sixth switch, and the eight switch includes a first resistor, a first transistor, a second resistor, and a second transistor; wherein
one terminal of the first resistor is connected to the controller, the other terminal of the resistor is connected to a base of the first transistor, a collector of the first transistor is connected to one terminal of the second resistor, an emitter of the first transistor is grounded, and the other terminal of the second resistor is connected to a base of the second transistor;
for the first switch, an emitter of the second transistor is connected to the power terminal of the main unit via the first current control unit, and a collector of the second transistor is connected to the first electrode connector;
for the third switch, the emitter of the second transistor is connected to the power terminal of the main unit via the second current control unit, and the collector of the second transistor is connected to the second electrode connector;
for the fourth switch, the emitter of the second transistor is connected to the second electrode connector, and the collector of the second transistor is grounded via the second current detection unit;
for the fifth switch, the emitter of the second transistor is connected to the power terminal of the main unit via the third current control unit, and the collector of the second transistor is connected to the third electrode connector;
for the sixth switch, the emitter of the second transistor is connected to the third electrode connector, and the collector of the second transistor is grounded via the third current detection unit; and
for the eighth switch, the emitter of the second transistor is connected to the fourth electrode connector, and the collector of the second transistor is grounded via the fourth current detection unit.

In some embodiments, each of the current detection units includes a third transistor, a third resistor, a fourth resistor, and a diode;
wherein a collector of the third transistor is connected to the controller, an emitter of the third transistor is grounded, a base of the third transistor is connected to both one terminal of the third resistor and one terminal of the fourth resistor, the other terminal of the third resistor is grounded, the other terminal of the fourth resistor is connected to both the other terminal of the third resistor and a positive terminal of the diode, and a negative terminal of the diode is connected to the electrode connector corresponding to the current detection unit.

In some embodiments, the main unit further includes a second switch and a seventh switch, wherein the first electrode connector is grounded via the second switch, the fourth electrode connector is connected to the power terminal of the main unit via the seventh switch; wherein
in a case where the first electrode connector forms a first path with the second electrode connector, the controller is configured to alternately control a first group of switches and a second group of switches to be turned on to supply a bidirectional pulse to the first path, wherein the first group of switches includes the first switch and the fourth switch, and the second group of switches includes the second switch and the third switch;
in a case where the first electrode connector forms a second path with the third electrode connector, the controller is configured to alternately control a third group of switches and a fourth group of switches to be turned on to supply a bidirectional pulse to the second path, wherein the third group of switches includes the first switch and the sixth switch, and the fourth group of switches includes the second switch and the fifth switch;
in a case where the first electrode connector forms a third path with the fourth electrode connector, the controller is configured to alternately control a fifth group of switches and a sixth group of switches to be turned on to supply a bidirectional pulse to the third path, wherein the fifth group of switches includes the first switch and the eighth switch, and the sixth group of switches includes the second switch and the seventh switch;
in a case where the second electrode connector forms a fourth path with the third electrode connector, the controller is configured to alternately control a seventh group of switches and an eighth group of switches to be turned on to supply a bidirectional pulse to the fourth path, wherein the seventh group of switches includes the third switch and the sixth switch, and the eighth group of switches includes the fourth switch and the fifth switch;
in a case where the second electrode connector forms a fifth path with the fourth electrode connector, the controller is configured to alternately control a ninth group of switches and a tenth group of switches to be turned on to supply a bidirectional pulse to the fifth path, wherein the ninth group of switches includes the third switch and the eighth switch, and the fourth group of switches includes the fourth switch and the seventh switch; and
in a case where the third electrode connector forms a sixth path with the fourth electrode connector, the controller is configured to alternately control an eleventh group of switches and a twelfth group of switches to be turned on to supply a bidirectional pulse to the sixth path, wherein the eleventh group of switches includes the fifth switch and the eighth switch, and the twelfth group of switches includes the sixth switch and the seventh switch.

In the embodiments of the present disclosure, a magnet magnetized along an axial direction of the electrode connector is provided at the electrode connector of the electrode lead for the therapeutic device. The insulating enclosure of the electrode connector includes two end faces, with the conductive portion of the electrode connector exposed at one end face for coupling with the electrode pads. The first region of the other end face is planar, and the contact surface of the conductive portion of the electrode connector is also planar. Through this design, any two electrode connectors may be magnetically attracted to each other, and a plurality of electrode connectors may be sequentially magnetically attracted and stacked. This prevents the electrode leads from tangling, reduces the risk of wire damages, and ensures that the wires are neatly organized during storage, such that the user experience is improved.

The above description only summarizes the technical solutions of the present disclosure. Specific embodiments of the present disclosure are described hereinafter to better and clearer understand the technical solutions of the present disclosure, to practice the technical solutions based on the disclosure of the specification, and to make the above and other objectives, features and advantages of the present disclosure more apparent and understandable.

### BRIEF DESCRIPTION OF THE DRAWINGS

By reading the detailed description of preferred embodiments hereinafter, various other advantages and beneficial effects become clear and apparent for persons of ordinary skill in the art. The accompanying drawings are merely for illustrating the preferred embodiments, but shall not be construed as limiting the present disclosure. In all the accompanying drawings, like reference numerals denote like parts. In the drawings:

FIG. 1A is a schematic structural diagram of a therapeutic device in the related art;

FIG. 1B is a schematic structural diagram of a therapeutic device according to some embodiments of the present disclosure;

FIG. 2 is a schematic structural diagram of a main unit and an electrode lead of a single-interface therapeutic device;

FIG. 3A is a schematic structural diagram of an electrode lead for a therapeutic device according to some embodiments of the present disclosure;

FIG. 3B is a schematic structural diagram of an electrode lead for a therapeutic device according to some embodiments of the present disclosure;

FIG. 3C is a schematic structural diagram of an electrode lead for a therapeutic device according to some embodiments of the present disclosure;

FIG. 4 is a schematic partial structural diagram of an electrode lead for a therapeutic device according to some embodiments of the present disclosure;

FIG. 5 is a schematic partial section view of an electrode lead for a therapeutic device according to some embodiments of the present disclosure;

FIG. 6 is a schematic partial exploded diagram of an electrode lead for a therapeutic device according to some embodiments of the present disclosure;

FIG. 7 is a schematic partial exploded diagram, from another perspective, of an electrode lead for a therapeutic device according to some embodiments of the present disclosure;

FIG. 8A is a schematic diagram of a pre-connection state of two electrode connectors;

FIG. 8B is a schematic diagram of a post-connection state of two electrode connectors;

FIG. 9A is a schematic diagram of a pre-connection state of four electrode connectors;

FIG. 9B is a schematic diagram of a post-connection state of four electrode connectors;

FIG. 10A is a schematic diagram of a pre-connection state of four electrode connectors;

FIG. 10B is a schematic diagram of a post-connection state of four electrode connectors;

FIG. 11 is a structural block diagram of a therapeutic device according to some embodiments of the present disclosure;

FIG. 12 is a schematic structural diagram of a therapeutic device according to some embodiments of the present disclosure;

FIG. 13 is a schematic structural diagram of a therapeutic device according to some embodiments of the present disclosure;

FIG. 14 is a schematic structural diagram of a therapeutic device according to some embodiments of the present disclosure;

FIG. 15A is a circuit diagram related to a first electrode connector in a path detection circuit of a therapeutic device according to some embodiments of the present disclosure;

FIG. 15B is a circuit diagram related to a second electrode connector in a path detection circuit of a therapeutic device according to some embodiments of the present disclosure;

FIG. 15C is a circuit diagram related to a third electrode connector in a path detection circuit of a therapeutic device according to some embodiments of the present disclosure;

FIG. 15D is a circuit diagram related to a fourth electrode connector in a path detection circuit of a therapeutic device according to some embodiments of the present disclosure;

FIG. 16 is a simplified diagram of a path detection circuit of a therapeutic device according to some embodiments of the present disclosure; and

FIG. 17 is a schematic diagram of a usage state of a therapeutic device according to some embodiments of the present disclosure.

Reference numerals in the embodiments and denotations thereof:
100, 200—therapeutic device; 1—safety socket; 2—USB interface; 3—type-C interface; 4, 20—electrode lead for therapeutic device; 10—main unit; 21—wire; 21a—first end of wire; 21b—second end of wire; 21a—first wire; 21b—second wire; 21c—third wire; 21d—fourth wire; 22—main unit connector; 23—electrode connector; 231—conductive portion; 2311—contact surface; 232—insulating enclosure; 232a—first end face; 232b—second end face; 2321—first housing; 2322—second housing; 2323—first engagement portion; 2324—second engagement portion; 2325—through hole; 2326—protrusion; 2327—recess; 233—magnet; 23a—first electrode connector; 23b—second electrode connector; 23c—third electrode connector; 23d—fourth electrode connector; 24—wired control module.

### DETAILED DESCRIPTION

The embodiments containing the technical solutions of the present disclosure are described in detail with reference to the accompanying drawings. The embodiments hereinafter are only used to clearly describe the technical solutions of the present disclosure. Therefore, these embodiments are only used as examples, but are not intended to limit the protection scope of the present disclosure.

A therapeutic apparatus (e.g., a portable transcutaneous electrical nerve stimulation (TENS) device) stimulates nerve fibers to activate a pain suppression mechanism of a human body by applying low-frequency or medium-frequency electrical pulses to human skin, including the gate control effect in the spinal cord and the promotion of the release of analgesic substances, such as endorphins. This mechanism helps alleviate or block transmission of pain signals to the brain, such that a therapeutic effect in pain relief is achieved.

The therapeutic device is primarily constituted by a main unit and electrode pads. The electrode pads are attached to painful areas of the human body, such as the neck, shoulders, back, waist, arms, legs, and other regions, and are connected to the main unit via electrode leads for the therapeutic device. Electrical pulse stimulation signals output by the main unit are transmitted to the human body via the electrode pads.

Electrode pads in the therapeutic device are typically used in pairs, allowing a current to pass through the body tissues and form a path to stimulate the nerve fibers, thereby alleviating pain. According to the number of output channels, the therapeutic device may be put into either single-channel use or dual-channel use. In single-channel use, the therapeutic device is connected to only one pair of electrode pads, and the current flows along a single specific path. For example, in a case where the user experiences back pain, the two electrode pads are placed near the painful area on the back. In dual-channel use, the therapeutic device provides two independent outputs, each connected to one pair of electrode pads. That is, the device is connected to two pairs of electrode pads, which allows simultaneous treatment of two different areas. For instance, one pair of electrode pads may be placed on the back, while the other pair may be placed on the legs. Current intensity, frequency, and waveform of each channel may be independently adjusted. Furthermore, therapeutic devices with three or more channels are also available on the market.

Since the main unit of the therapeutic device requires the use of at least two electrode pads, it is necessary to equip the device with a plurality of electrode leads to establish connections between the main unit and the electrode pads. In a case where the number of electrode leads of the therapeutic device is large, the wires are prone to entangling with each other, which may damage the wires and cause the wires to become disorganized and untidy. This results in inconvenience for a user when organizing the wires, leading to a poor user experience.

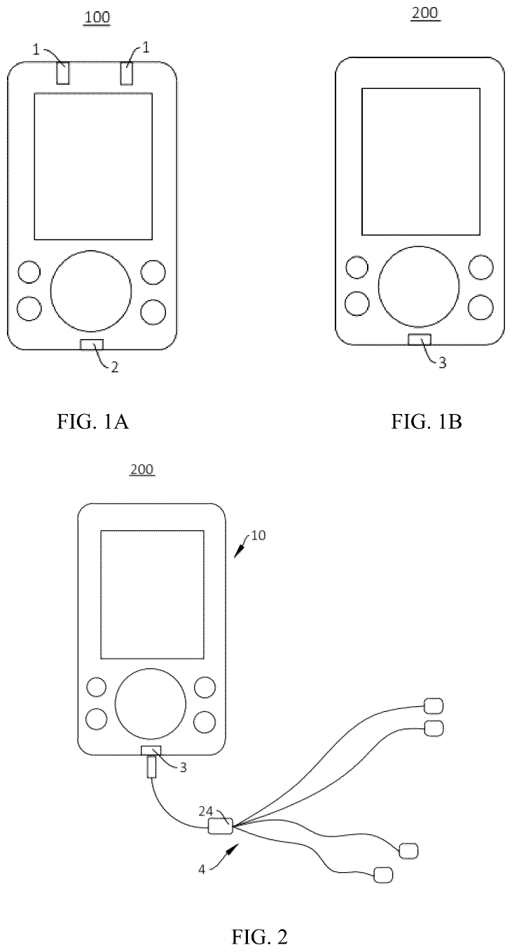

FIG. 1A and FIG. 1B are schematic structural diagrams of a therapeutic device 100 and a therapeutic device 200 with two types of interface designs. As illustrated in FIG. 1A, the therapeutic device 100 employs a safety socket 1 as an output interface of the electrical pulse signal from the therapeutic device. Additionally, a USB interface 2 (or other power sockets) is included as a charging interface, that is, the device is configured with two types of interfaces. In a case where a user utilizes the therapy function while the therapeutic device 100 is in a charging state, i.e., the charging interface and the output interface are used simultaneously, the charging interface and the electrode lead(s) connected to the therapeutic device are in the same circuit. Consequently, the mains power and the human body are in the same circuit, which may present safety hazards. To avoid the risk of the mains power and the human body being in the same circuit during the use of the therapeutic device 100, as illustrated in FIG. 1B, in the therapeutic device 200 according to some embodiments of the present disclosure, the output interface and the charging interface are merged into a single interface, for example, a type-C interface 3. The type-C interface 3 may only be connected to either a charging cable or an electrode lead at any one time. This prevents the safety risk associated with a user operating the therapeutic device 200 while the device is in the charging state. Hereinafter, the therapeutic device 200 illustrated in FIG. 1B may be referred to as a single-interface therapeutic device.

FIG. 2 is a schematic structural diagram of a main unit and lead(s) of a single-interface therapeutic device. As illustrated in FIG. 2, for a dual-channel single-interface therapeutic device 200, only a single electrode lead 4 with four electrode connectors needs to be configured, which reduces cost and improves portability. However, with such a electrode lead 4, the issue of the wires easily becoming tangled still exists. FIG. 2 illustrates a dual-channel single-interface therapeutic device 200 merely as an example for description. For a tri-channel therapeutic device or a therapeutic device with more than three channels (i.e., higher channel counts), the number of wires is greater, and thus wire tangling occur more likely.

As illustrated in FIG. 2, a wired control module 24 is arranged on the wire(s) of the electrode lead 4, wherein the wired control module 24 is configured to perform one or more of the following functions: selecting a treatment mode, adjusting a current intensity, adjusting a treatment frequency, controlling a treatment time, and starting/stopping treatment. Alternatively, the electrode lead 4 may not be provided with the wired control module 24, and instead the aforementioned functions are performed by operating the main unit 10.

Accordingly, the present disclosure provides an electrode lead for a therapeutic device. A magnet magnetized along an axial direction of the electrode connector is provided at the electrode connector of the electrode lead for the therapeutic device. The insulating enclosure of the electrode connector includes two end faces, with the conductive portion of the electrode connector exposed at one end face for coupling with the electrode pads. The first region of the other end face is planar, and the contact surface of the conductive portion of the electrode connector is also planar. Through this design, any two electrode connectors may be magnetically attracted to each other, and a plurality of electrode connectors may be sequentially magnetically attracted and stacked. This prevents the electrode leads from tangling, reduces the risk of wire damages, and ensures that the wires are neatly organized during storage, such that the user experience is improved.

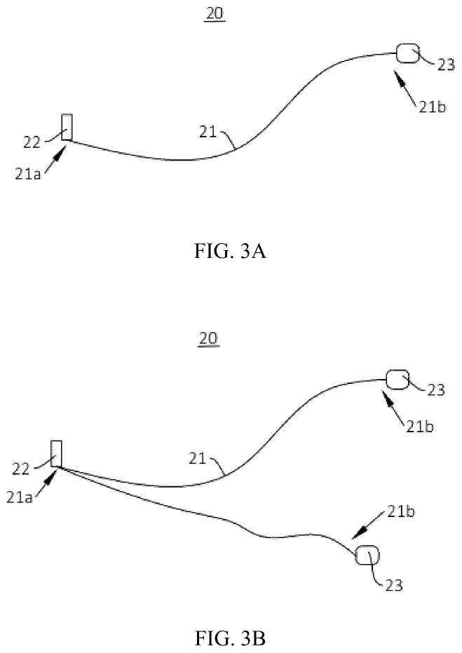

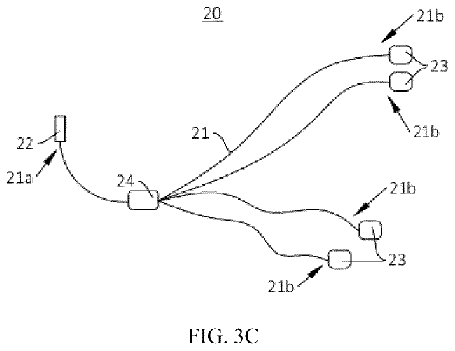

FIG. 3A and FIG. 3B are structural schematic diagrams of several electrode leads 20 for a therapeutic device according to some embodiments of the present disclosure. As illustrated in FIG. 3A and FIG. 3B, each of the electrode leads 20 includes at least one wire 21, a main unit connector 22, and at least one electrode connector 23.

The electrode lead 20 illustrated in FIG. 3A includes one wire 21 and one electrode connector 23. The main unit connector 22 is a connector configured to mate with the safety socket 1 illustrated in FIG. 1A. The electrode lead 20 is suitable for use with the therapeutic device 100 in FIG. 1A configured with two interfaces.

The electrode lead 20 illustrated in FIG. 3B includes two wires 21 and two electrode connectors 23. The main unit connector 22 may be a connector configured to mate with the safety socket 1 illustrated in FIG. 1A. In this case, the electrode lead 20 is suitable for use with the therapeutic device 100 in FIG. 1A configured with two interfaces. Alternatively, the main unit connector 22 may be a connector configured to mate with the type-C interface 3 illustrated in FIG. 1B. In this case, the electrode lead 20 is suitable for use with the therapeutic device 200 in FIG. 1B which has a type-C interface.

The electrode lead 20 illustrated in FIG. 3C includes four wires 21 and four electrode connectors 23. The main unit connector 22 is a connector configured to mate with the type-C interface 3 illustrated in FIG. 1B. This electrode lead 20 is suitable for use with the therapeutic device 200 in FIG. 1B which has a type-C interface.

In a case where the main unit connector 22 is configured to mate with the type-C interface 3, an all-plastic type-C male connector may be employed to be plugged into the type-C female connector of the main unit 10. Compared to the conventional approach of using a metal male connector with an added insulating sleeve, the all-plastic type-C male connector is thinner, lighter, and more aesthetically pleasing.

The electrode lead 20 according to the present disclosure may also include wires 21 and electrode connectors 23 in another quality, such as 3, 5, 6, 7, 8, or the like. FIG. 3A to FIG. 3C are illustrative only and do not limit the numbers of wires 21 and electrode connectors 23 of the present disclosure.

Referring further to FIG. 3A and FIG. 3B, the main unit connector 22 and the electrode connector 23 are respectively disposed at two ends of the wire 21, wherein the main unit connector 22 is disposed at a first end 21a of the wire, and the electrode connector 23 is disposed at a second end 21b of the wire. During use of the therapeutic device 200, the main unit connector 22 is electrically connected to the main unit 10 of the therapeutic device 200, the electrode connector 23 is connected to an electrode pad, and the electrode pad is attached to the skin of the user. An electrical pulse stimulation signal output by the main unit 10 is transmitted sequentially through the main unit connector 22, the wire 21, the electrode connector 23, and the electrode pad to the human body.

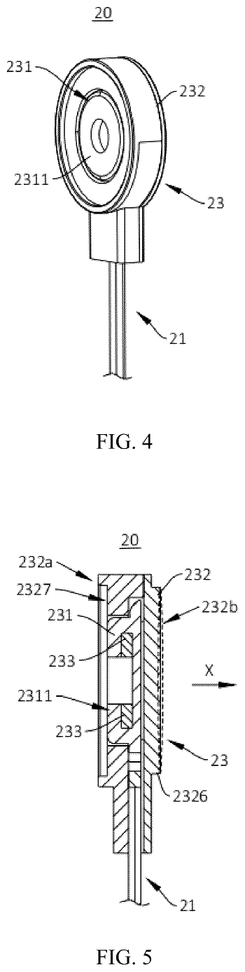

FIG. 4 is a schematic partial structural diagram of an electrode lead according to some embodiments of the present disclosure. Only the electrode connector 23 and a portion of the wire 21 are illustrated in FIG. 4, and the main unit connector is not illustrated. As illustrated in FIG. 4, the electrode connector 23 includes a conductive portion 231 and an insulating enclosure 232. The insulating enclosure 232 may be fabricated by injection molding.

The conductive portion 231 is electrically connected to the main unit connector via the wire 21. The conductive portion 231 includes a contact surface 2311 for connecting to an electrode pad. The insulating enclosure 232 covers the conductive portion 231, with the contact surface 2311 being exposed, such that the contact surface 2311 is connected to the electrode pad. The connection between the contact surface 2311 and the electrode pad may be a snap-fit (engagement) connection or a magnetic connection. The present disclosure uses magnetic connection as an example for description.

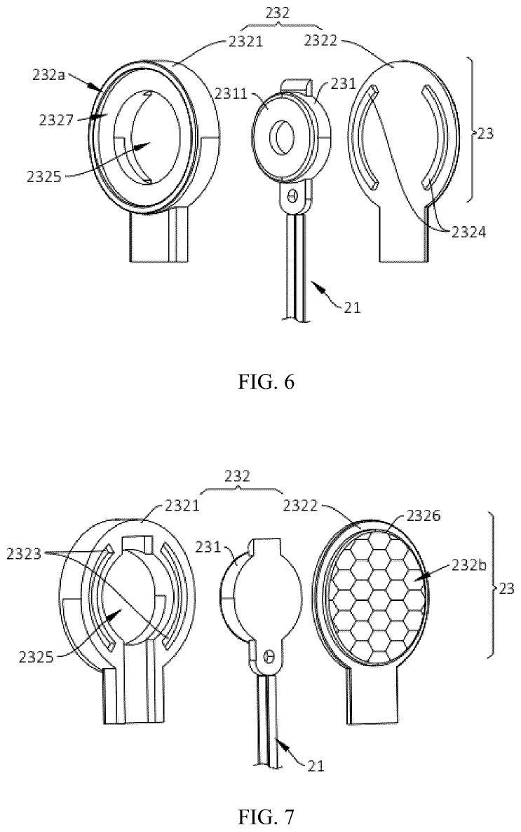

FIG. 5 is a schematic partial section view of an electrode lead for a therapeutic device according to some embodiments of the present disclosure. FIG. 6 is a schematic partial exploded diagram of an electrode lead for a therapeutic device according to some embodiments of the present disclosure. FIG. 7 is a schematic partial exploded diagram, from another perspective, of an electrode lead for a therapeutic device according to some embodiments of the present disclosure; As illustrated in FIG. 5 to FIG. 7, a magnet 233 is disposed within the conductive portion 231. The magnet 233 is magnetized along an axial direction thereof, i.e., magnetized along a first direction X illustrated in the drawings. The axial direction (first direction X) of the magnet 233 is perpendicular to the contact surface 2311. The insulating enclosure 232 includes a first end face 232a and a second end face 232b opposite each other along the first direction X. The contact surface 2311 is exposed at the first end face 232a. A first region within the second end face 232b (a region enclosed by the dashed circle in FIG. 5 and the honeycomb-filled region in FIG. 7) and the contact surface 2311 are both planar. This allows the first region to be magnetically connected to the contact surface 2311 of another electrode connector in a case where the electrode lead 20 is in a non-use state, such that storage of the electrode lead 20 is facilitated. In a case where the first region and the contact surface 2311 are non-planar, for example, curved surfaces, the first region may be prevented from being magnetically connected to the contact surface 2311 of another electrode connector.

In a case where the electrode lead 20 includes a plurality of electrode connectors 23, mounting orientations of the magnetic poles of the magnet 233 in each electrode connector 23 are consistent. For example, the N pole of the magnet 233 in each electrode connector 23 is close to the first end face 232a, and the S pole is close to the second end face 232b; alternatively, the N pole of the magnet 233 in each electrode connector 23 is close to the second end face 232b, and the S pole is close to the first end face 232a. In this way, two electrode connectors 23 arranged in the same relative orientation may be magnetically connected to each other. Otherwise, in a case where the two electrode connectors 23 are arranged together in the same relative orientation, magnetic repulsion between like poles would prevent these poles from attaching to each other.

The insulating enclosure 232 includes a first housing 2321 and a second housing 2322, wherein a first engagement portion 2323 is arranged on the first housing 2321, and a second engagement portion 2324 is arranged on the second housing 2322, wherein the first engagement portion 2323 and the second engagement portion 2324 are engaged with each other to secure the first housing 2321 and the second housing 2322. By providing two housing and engaging (snap-fitting) the two housing together to form the insulating enclosure 232, the assembly operation is facilitated.

The first engagement portion 2323 and the second engagement portion 2324 may be respectively a recess or a protrusion. For example, the first engagement portion 2323 is an arcuate recess, and the second engagement portion 2324 is an arcuate protrusion, wherein the arcuate recess is adapted to the arcuate protrusion in terms of shape and size. A plurality of arcuate recess and a plurality of arcuate protrusions may be arranged. In the embodiments as illustrated in the drawings, two arcuate recess and two arcuate protrusions are arranged. Alternatively, the first engagement portion 2323 is an arcuate protrusion, and the second engagement portion 2324 is an arcuate recess. In this way, engagement between the arcuate grooves and the arcuate protrusions are more stable and secure.

A through hole 2325 is defined in the first housing 2321, wherein the through hole 2325 is configured to accommodate the conductive portion 231. During assembly, the conductive portion 231 is placed into the through hole 2325, and then the first housing 2321 and the second housing 2322 are engaged with each other.

A first end face 232a of the insulating enclosure 232 is disposed on the first housing 2321, and a second end face 232b of the insulating enclosure 232 is disposed on the second housing 2322. A protrusion 2326 is arranged on the second end surface 232b of the insulating enclosure 232, wherein a top surface of the protrusion 2326 forms the first region; and a recess 2327 is defined in the first end surface 232a of the insulating enclosure 232, wherein a shape and size of the recess 2327 are configured to be adapted for engagement within a protrusion 2326 of another electrode connector 23. As illustrated in the drawings, the protrusion 2326 and the recess 2327 both have circular cross-sections, with circular diameters thereof substantially the same. The circular diameter of the recess 2327 may be slightly greater than the circular diameter of the protrusion 2326, such that in a case where two electrode connectors 23 are magnetically attached, the protrusion 2326 of one electrode connector 23 are insertable into the recess 2327 of the other electrode connector 23. The circular cross-sections of the protrusion 2326 and the recess 2327 facilitate their engagement (or snap-fitting). In a case where the two electrode connectors 23 are magnetically attached, the protrusion 2326 may be inserted into the recess 2327 at any relative angle between the two electrode connectors 23, with no need to deliberately place the two electrode connectors 23 at a specific angle. Nevertheless, the cross-sections of the protrusion 2326 and the recess 2327 may also be other shapes, for example, square, triangle, ellipse, or the like.

A bottom surface of the recess 2327 is flush with the contact surface 2311, and a height of the protrusion 2326 is equal to a depth of the recess 2327. In a case where the two electrode connectors 23 are magnetically attached, the protrusion 2326 of one electrode connector 23 may fit precisely into the recess 2327 of the other electrode connector 23, such that seamless magnetic attachment is achieved. Furthermore, the overall size after the two electrode connectors 23 are magnetically connected is relatively small, such that the required storage space is reduced.

The magnet 233 is configured as a ring-shaped (annular) body. An annular magnet concentrates the magnetic field, reduces magnetic leakage, and provides a more uniform magnetic field in its central region.

According to the present disclosure, the number of wires 21 is n, and the number of electrode connectors 23 is also n, wherein n is a positive integer greater than 1, and the n electrode connectors 23 are electrically connected to the main body connector 22 via the n wires 21 respectively. The electrode lead 20 for the therapeutic device is configured such that: in a non-use state, the n electrode connectors 23 are disposed adjacently, and in any adjacent two of the n electrode connectors 23, a contact surface 2311 of one electrode connector 23 is magnetically attached to a first region of the other electrode connector 23 such that the electrode lead 20 for the therapeutic device is stored.

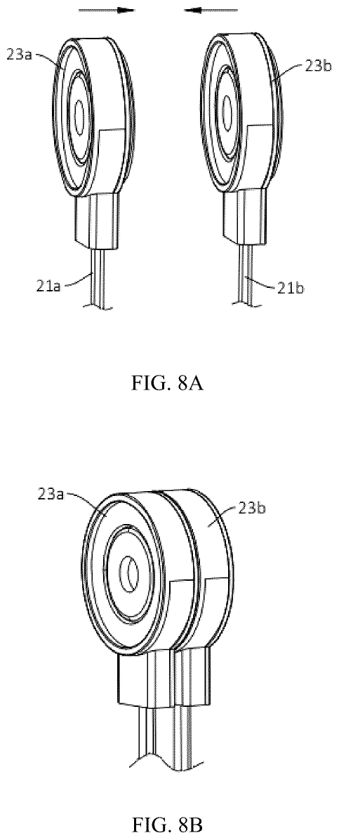

FIG. 8A is a schematic diagram of a pre-connection state of two electrode connectors, and FIG. 8B is a schematic diagram of a post-connection state of two electrode connectors. As illustrated in FIG. 8A, the wires include a first wire 21a and a second wire 21b, and the electrode connectors include a first electrode connector 23a and a second electrode connector 23b, wherein conductive portions of the first electrode connector 23a and the second electrode connector 23b are electrically connected to the main unit connector 22 via the first wire 21a and the second wire 21b respectively. In a non-use state, the first electrode connector 23a and the second electrode connector 23b are moved toward each other along a direction indicated by the arrow in FIG. 8A to achieve the state in FIG. 8B. In this case, a contact surface of the first electrode connector 23a is magnetically connected to a first region of the second electrode connector 23b, such that storage of the electrode lead is facilitated.

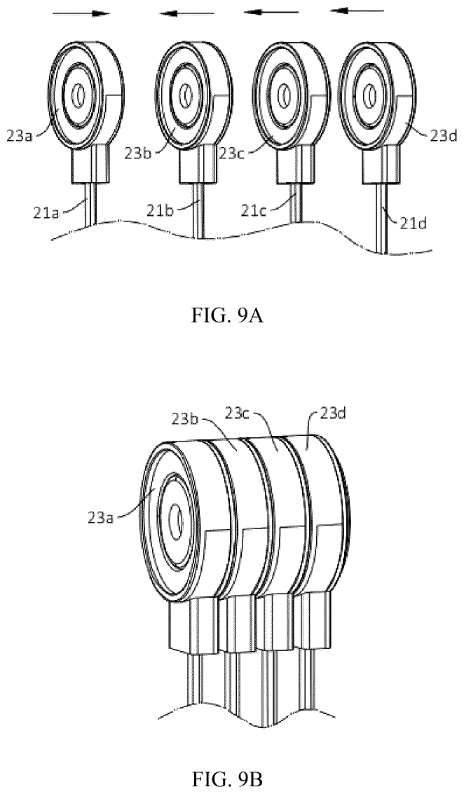

FIG. 9A is a schematic diagram of a pre-connection state of four electrode connectors, and FIG. 9B is a schematic diagram of a post-connection state of four electrode connectors. As illustrated in FIG. 9A, the wires include a first wire 21a, a second wire 21b, a third wire 21c, and a fourth wire 21d, and the electrode connectors include a first electrode connector 23a, a second electrode connector 23b, a third electrode connector 23c, and a fourth electrode connector 23d, wherein conductive portions of the first electrode connector 23a, the second electrode connector 23b, the third electrode connector 23c, and the fourth electrode connector 23d are electrically connected to the main unit connector via the first wire 21a, the second wire 21b, the third wire 21c, and the fourth wire 21d respectively. In a non-use state, the four electrode connectors are moved toward each other along a direction indicated by the arrow in FIG. 9A to achieve the state in FIG. 9B. In this case, a contact surface of the first electrode connector 23a is magnetically attached to a first region of the second electrode connector 23b, a contact surface of the second electrode connector 23b is magnetically attached to a first region of the third electrode connector 23c, and a contact surface of the third electrode connector 23c is magnetically attached to a first region of the fourth electrode connector 23d, such that the electrode lead for the therapeutic device is stored.

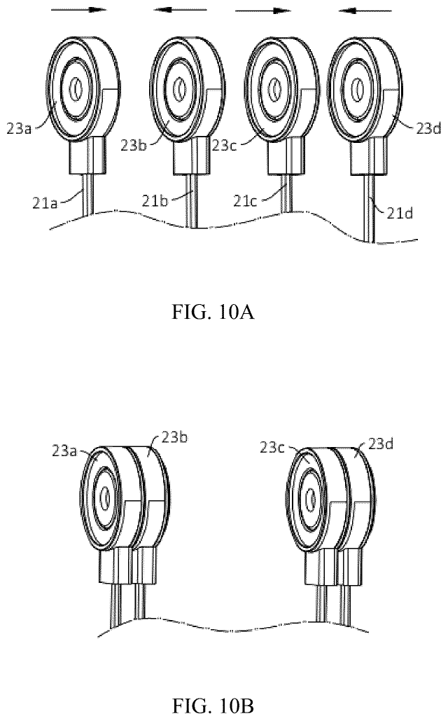

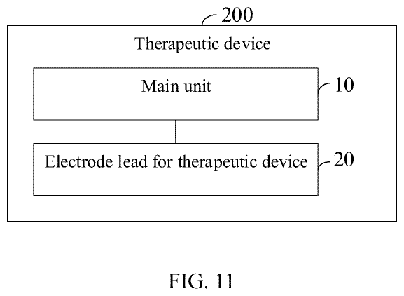

FIG. 10A is a schematic diagram of a pre-connection state of four electrode connectors, and FIG. 10B is a schematic diagram of a post-connection state of four electrode connectors. Referring to FIG. 10A, the four electrode connectors are moved toward each other along a direction indicated by the arrow in a direction indicated by the arrow in FIG. 10A to achieve the state in FIG. 10B. In this case, a contact surface of the first electrode connector 23a is magnetically attached to a first region of the second electrode connector 23b, and a contact surface of the third electrode connector 23c is magnetically attached to a first region of the fourth electrode connector 23d, such that a pair of electrode connectors for single-channel use is obtained. That is, the first electrode connector 23a and the second electrode connector 23b form one electrode connector of a pair of electrode connectors for single-channel use, and the third electrode connector 23c and the fourth electrode connector 23d form the other electrode connector of the pair of electrode connector for single-channel use.

In summary, with the electrode lead for the therapeutic device according to the embodiments of the present disclosure, the electrode connectors may be stacked and magnetically attached together in a non-use state, such that the electrode lead is prevented from tangling and becoming disorganized. In a use state, for example, for an electrode lead having four wires and four electrode connectors, by magnetically attaching the electrode connectors together in pairs, pairs of electrode connectors suitable for single-channel use are formed, such that some wires are prevented from being disorganized during use.

FIG. 11 is a structural block diagram of a therapeutic device according to some embodiments of the present disclosure. As illustrated in FIG. 11, the therapeutic device 200 includes the electrode lead 20 and the main unit 10 according to any of the above embodiments.

In some embodiments, the number of the at least one wire in the electrode lead 20 is n, and the number of the at least one electrode connector is also n, wherein n is a positive integer greater than 1. The main unit includes a controller, n switch units, and n current detection units, wherein the n switch units are in one-to-one correspondence with the n electrode connectors, the n current detection units are in one-to-one correspondence with the n electrode connectors, the n electrode connectors include a first electrode connector, the n switch units include a first switch unit in correspondence with the first electrode connector, and the n current detection units include a first current detection unit in correspondence with the first electrode connector, wherein the first switch unit is connected to a power terminal of the main unit, and the other switches in the n switch units are grounded. The controller is configured to control the first switch unit to be turned on and control a target switch unit in the other switch units to be turned on, and detect a current at the first electrode connector via the first current detection unit or detect a current at a target electrode connector corresponding to a target current detection unit in the n current detection units via the target current detection unit to determine whether a path is formed between the first electrode connector and the target electrode connector, wherein the target electrode connector is in correspondence with the target switch unit.

The controller may be a microcontroller, a microcontroller unit (MCU), a programmable logic controller (PLC), a digital signal processor (DSP), a field-programmable gate array (FPGA), a system on chip (SoC), or the like, preferably an MCU.

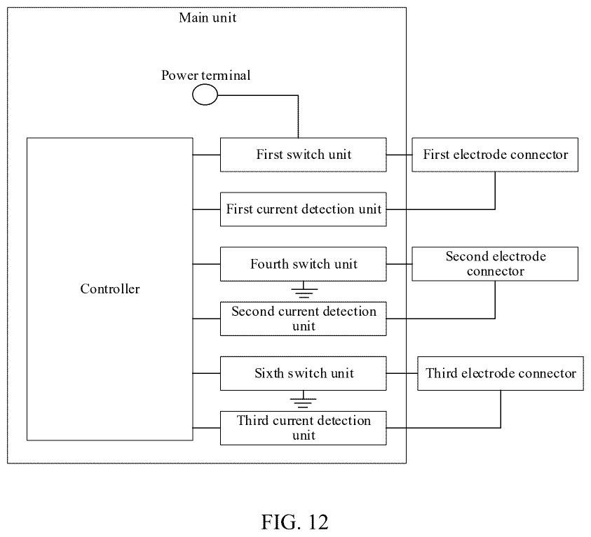

FIG. 12 is a schematic structural diagram of a therapeutic device according to some embodiments of the present disclosure. Wires of therapeutic device include a first wire, a second wire, and a third wire. As illustrated in FIG. 12, electrode connectors of the therapeutic device include a first electrode connector, a second electrode connector, and a third electrode connector.

The main unit includes a controller, a first switch unit, a fourth switch unit, a sixth switch unit, a first current detection unit, a second current detection unit, and a third current detection unit. The first switch unit, the fourth switch unit, and the sixth switch unit are in-one-to-one correspondence with and connected to the first electrode connector, the second electrode connector, and the third electrode connector. The first current detection unit, the second current detection unit, and the third current detection unit are in-one-to-one correspondence with and respectively connected to the first electrode connector, the second electrode connector, and the third electrode connector. The switch units and the current detection units are all connected to the controller. The first switch unit is connected to the power terminal of the main unit, and the fourth switch unit and the sixth switch unit are grounded.

The controller is configured to control the first switch unit to be turned on and control a target switch unit in the fourth switch unit and the sixth switch unit to be turned on, and detect a current at the first electrode connector via the first current detection unit or detect a current at a target electrode connector corresponding to a target current detection unit in the second current detection unit and the third current detection unit via the target current detection unit to determine whether a path is formed between the first electrode connector and the target electrode connector, wherein the target electrode connector is in correspondence with the target switch unit.

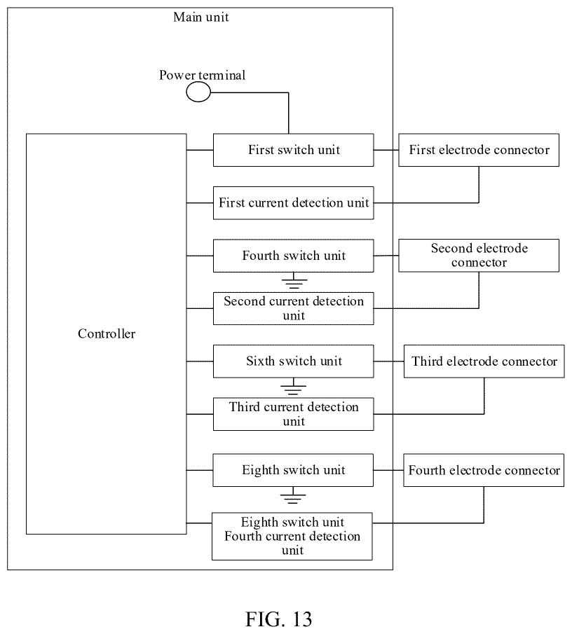

FIG. 13 is a schematic structural diagram of another therapeutic device according to some embodiments of the present disclosure. Wires of the therapeutic device include a first wire, a second wire, and a third wire. As illustrated in FIG. 13, electrode connectors of the therapeutic device include a first electrode connector, a second electrode connector, a third electrode connector, and a fourth electrode connector.

The main unit includes a controller, a first switch unit, a fourth switch unit, a sixth switch unit, an eighth switch unit, a first current detection unit, a second current detection unit, a third current detection unit, and a fourth current detection unit. The first switch unit, the fourth switch unit, the sixth switch unit, and the eighth switch unit are in-one-to-one correspondence with and respectively connected to the first electrode connector, the second electrode connector, the third electrode connector, and the fourth electrode connector. The first current detection unit, the second current detection unit, the third current detection unit, and the fourth current detection unit are in one-to-one correspondence with and respectively connected to the first electrode connector, the second electrode connector, the third electrode connector, and the fourth electrode connector. The first switch unit is connected to the power terminal of the main unit, and the fourth switch unit, the sixth switch unit, and the eighth switch unit are grounded.

The controller is configured to control the first switch unit to be turned on and control a target switch unit in the fourth switch unit, the sixth switch unit, and the eight switch unit to be turned on, and detect a current at the first electrode connector via the first current detection unit or detect a current at a target electrode connector corresponding to a target current detection unit in the second current detection unit, the third current detection unit, and the fourth current detection unit via the target current detection unit to determine whether a path is formed between the first electrode connector and the target electrode connector, wherein the target electrode connector is in correspondence with the target switch unit.

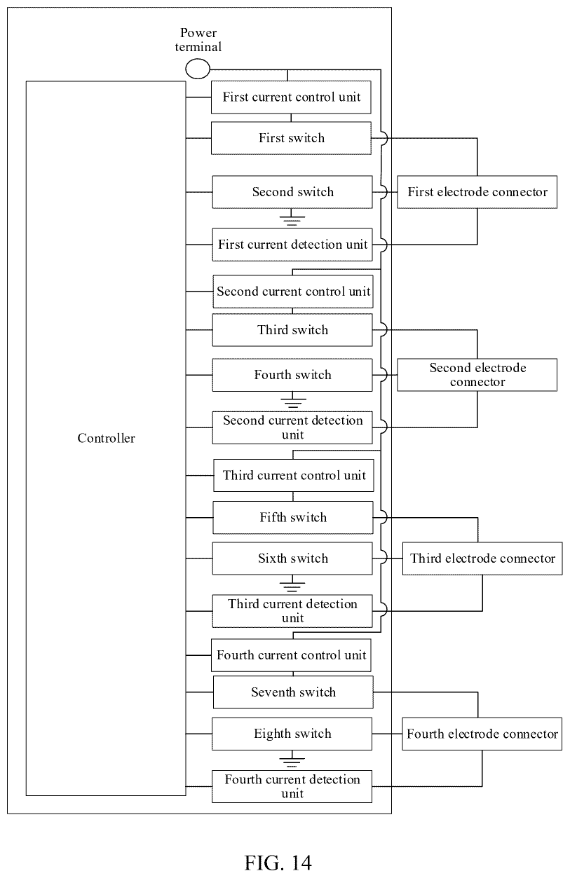

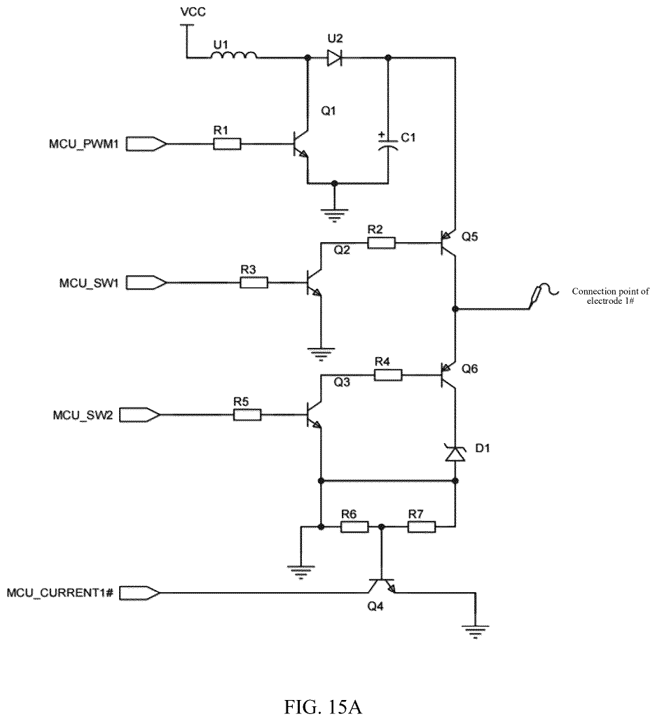

FIG. 14 is a schematic structural diagram of another therapeutic device according to some embodiments of the present disclosure. Wires of the therapeutic device include a first wire, a second wire, and a third wire. As illustrated in FIG. 14, electrode connectors of the therapeutic device include a first electrode connector, a second electrode connector, a third electrode connector, and a fourth electrode connector.

The main unit includes a controller, a first switch, a third switch, a fourth switch, a fifth switch, a sixth switch, an eighth switch, a first current detection unit, a second current detection unit, a third current detection unit, and a fourth current detection unit.

The first electrode connector is connected to the power terminal of the main unit via the first switch, the second electrode connector is connected to the power terminal of the main unit via the third switch and further grounded via the fourth switch, the third electrode connector is connected to the power terminal of the main unit via the fifth switch and further grounded via the sixth switch, and the fourth electrode connector is grounded via the eighth switch.

The first current detection unit, the second current detection unit, the third current detection unit, and the fourth current detection unit are respectively connected to the first electrode connector, the second electrode connector, the third electrode connector, and the fourth electrode connector.

The controller is configured to:
control the first switch and the fourth switch to be turned on, and detect a current at the first electrode connector via the first current detection unit or detect a current at the second electrode connector via the second current detection circuit to determine whether a path is formed between the first electrode connector and the second electrode connector;
control the first switch and the sixth switch to be turned on, and detect a current at the first electrode connector via the first current detection unit or detect a current at the third electrode connector via the third current detection circuit to determine whether a path is formed between the first electrode connector and the third electrode connector;
control the first switch and the eighth switch to be turned on, and detect a current at the first electrode connector via the first current detection unit or detect a current at the fourth electrode connector via the fourth current detection circuit to determine whether a path is formed between the first electrode connector and the fourth electrode connector;
control the third switch and the sixth switch to be turned on, and detect a current at the second electrode connector via the second current detection unit or detect a current at the third electrode connector via the third current detection circuit to determine whether a path is formed between the second electrode connector and the third electrode connector;
control the third switch and the eighth switch to be turned on, and detect a current at the second electrode connector via the second current detection unit or detect a current at the fourth electrode connector via the fourth current detection circuit to determine whether a path is formed between the second electrode connector and the fourth electrode connector; or
control the fifth switch and the eighth switch to be turned on, and detect a current at the third electrode connector via the third current detection unit or detect a current at the fourth electrode connector via the fourth current detection circuit to determine whether a path is formed between the third electrode connector and the fourth electrode connector.

Referring to FIG. 14, a first current control unit is further connected between the first switch and the power terminal of the main unit; and in a case where the first electrode connector forms a first path, a second path, or a third path with the second electrode connector, the third electrode connector, or the fourth electrode connector, the controller is configured to control a magnitude of current of the first path, the second path, or the third path via the first current control unit.

A second current control unit is further connected between the third switch and the power terminal of the main unit; and in a case where the first electrode connector forms a fourth path or a fifth path with the third electrode connector or the fourth electrode connector, the controller is configured to control a magnitude of current of the fourth path or the fifth path via the second current control unit.

A third current control unit is further connected between the fourth switch and the power terminal of the main unit; and in a case where the third electrode connector forms a sixth path with the fourth electrode connector, the controller is configured to control a magnitude of current of the sixth path via the third current control unit.

Referring to FIG. 14, the main unit further includes a second switch and a seventh switch, wherein the first electrode connector is grounded via the second switch, the fourth electrode connector is connected to the power terminal of the main unit via the seventh switch.

In a case where the first electrode connector forms a first path with the second electrode connector, the controller is configured to alternately control a first group of switches and a second group of switches to be turned on to supply a bidirectional pulse to the first path, wherein the first group of switches includes the first switch and the fourth switch, and the second group of switches includes the second switch and the third switch.

In a case where the first electrode connector forms a second path with the third electrode connector, the controller is configured to alternately control a third group of switches and a fourth group of switches to be turned on to supply a bidirectional pulse to the second path, wherein the third group of switches includes the first switch and the sixth switch, and the fourth group of switches includes the second switch and the fifth switch.

In a case where the first electrode connector forms a third path with the fourth electrode connector, the controller is configured to alternately control a fifth group of switches and a sixth group of switches to be turned on to supply a bidirectional pulse to the third path, wherein the fifth group of switches includes the first switch and the eighth switch, and the sixth group of switches includes the second switch and the seventh switch.

In a case where the second electrode connector forms a fourth path with the third electrode connector, the controller is configured to alternately control a seventh group of switches and an eighth group of switches to be turned on to supply a bidirectional pulse to the fourth path, wherein the seventh group of switches includes the third switch and the sixth switch, and the eighth group of switches includes the fourth switch and the fifth switch.

In a case where the second electrode connector forms a fifth path with the fourth electrode connector, the controller is configured to alternately control a ninth group of switches and a tenth group of switches to be turned on to supply a bidirectional pulse to the fifth path, wherein the ninth group of switches includes the third switch and the eighth switch, and the fourth group of switches includes the fourth switch and the seventh switch.

In a case where the third electrode connector forms a sixth path with the fourth electrode connector, the controller is configured to alternately control an eleventh group of switches and a twelfth group of switches to be turned on to supply a bidirectional pulse to the sixth path, wherein the eleventh group of switches includes the fifth switch and the eighth switch, and the twelfth group of switches includes the sixth switch and the seventh switch.

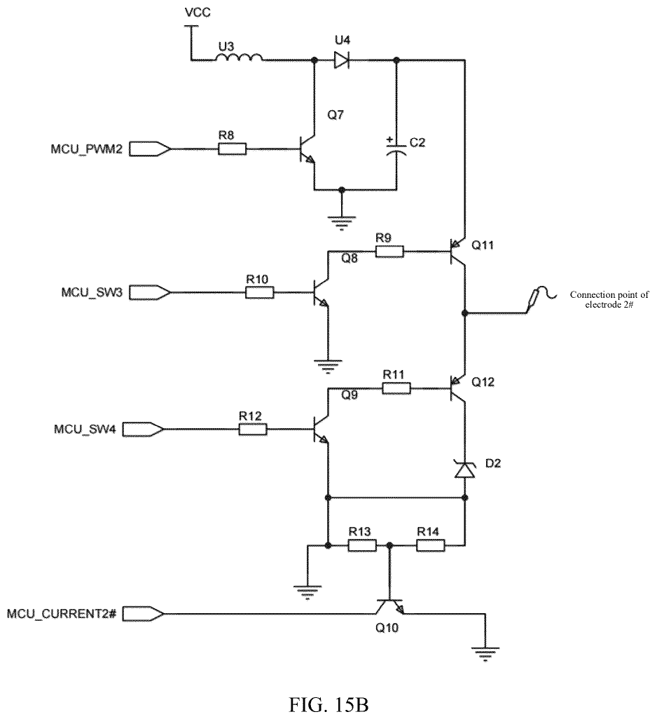

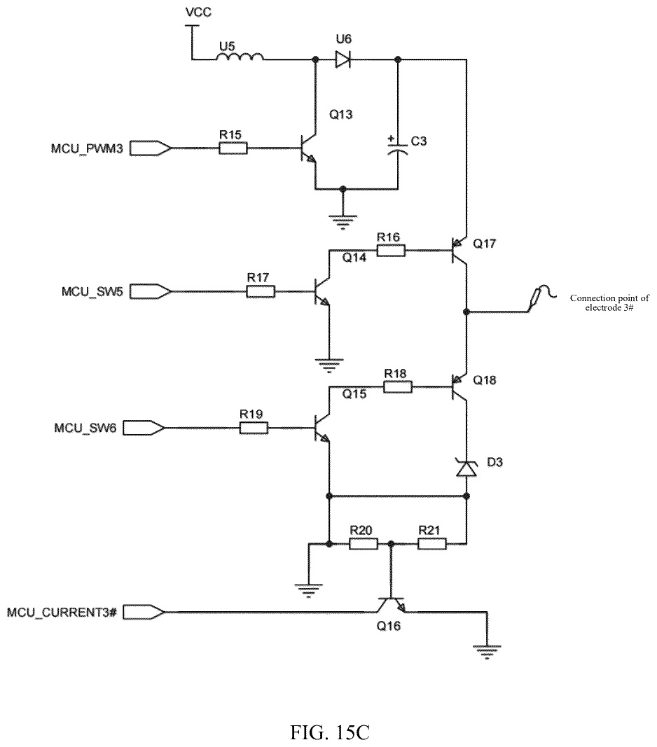

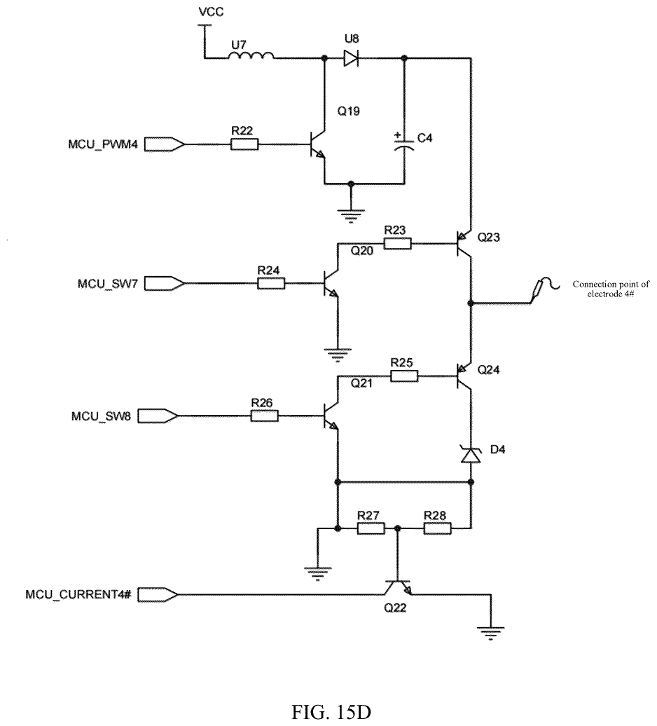

FIG. 15A to FIG. 15D are circuit diagrams of a path detection circuit of a therapeutic device according to some embodiments of the present disclosure, wherein description is given using a lead wire including four wires and four electrode connectors as an example. FIG. 15A illustrates a circuit related to a first electrode connector (labeled electrode 1 #), FIG. 15B illustrates a circuit related to a second electrode connector (labeled electrode 2 #) , FIG. 15C illustrates a circuit related to a third electrode connector (labeled electrode 3 #), and FIG. 15D illustrates a circuit related to a fourth electrode connector (labeled electrode 4 #).

As illustrated in FIG. 15A to FIG. 15D, each switch is composed of the same circuit devices or elements. Using a first switch as an example, the same structures and connections of the switches are described. As illustrated in FIG. 15A, the first switch includes a first resistor R3, a first transistor Q2, a second resistor R2, and a second transistor Q5. One terminal of the first resistor R3 is connected to the controller (connected to an MCU_SW1 pin of the controller), the other terminal of the first resistor R3 is connected to a base of the first transistor Q2, a collector of the first transistor Q2 is connected to one terminal of the second resistor R2, an emitter of the first transistor Q2 is grounded, and the other terminal of the second resistor R2 is connected to a base of the second transistor Q5.

As illustrated in FIG. 15A, for the first switch, an emitter of the second transistor Q5 is connected to the power terminal VCC of the main unit via the first current control unit, and a collector of the second transistor Q5 is connected to the first electrode connector (electrode 1 #). For the second switch, an emitter of a second transistor Q6 is connected to the first electrode connector (electrode 1 #), and a collector of the second transistor Q6 is grounded via the second current detection unit.

As illustrated in FIG. 15B, for the third switch, an emitter of a second transistor Q11 is connected to the power terminal VCC of the main unit via the second current control unit, and a collector of the second transistor Q11 is connected to the second electrode connector (electrode 2 #). For the fourth switch, an emitter of a second transistor Q12 is connected to the second electrode connector (electrode 2 #), and a collector of the second transistor Q12 is grounded via the second current detection unit.

As illustrated in FIG. 15C, for the fifth switch, an emitter of a second transistor Q17 is connected to the power terminal VCC of the main unit via the third current control unit, and a collector of the second transistor Q17 is connected to the third electrode connector (electrode 3 #). For the sixth switch, an emitter of a second transistor Q18 is connected to the third electrode connector (electrode 3 #), and a collector of the second transistor Q18 is grounded via the third current detection unit.

As illustrated in FIG. 15D, for the seventh switch, an emitter of a second transistor Q23 is connected to the power terminal VCC of the main unit via the third current control unit, and a collector of the second transistor Q23 is connected to the fourth electrode connector (electrode 4 #). For the eighth switch, an emitter of a second transistor Q24 is connected to the fourth electrode connector (electrode 4 #), and a collector of the second transistor Q24 is grounded via the fourth current detection unit.

Regarding the working principle of the first switch, the controller controls, based on an MCU_SW1 signal, the first transistor Q2 to be turned on or turned off. In a case where the first transistor Q2 is turned on, the base of the second transistor Q5 receives a current through the second resistor R2, and the second transistor Q5 is turned on, such that the connection point of electrode 1 # is connected to the power terminal VCC. In a case where the first transistor Q2 is turned off, the second transistor Q5 is turned off, such that the connection point of electrode 1 # is disconnected from the power terminal VCC. The first resistor R3 achieves the function of current limiting and protection, such that the first transistor Q2 is ensured to operate safely and stably under control of a control signal from the controller.

Regarding the working principle of the second switch, the controller controls, based on an MCU_SW2 signal, a first transistor Q3 to be turned on or turned off. In a case where the first transistor Q3 is turned on, a base of the second transistor Q6 receives a current through a second resistor R4, and the second transistor Q6 is turned on, such that the connection point of electrode 1 # is grounded. In a case where the first transistor Q3 is turned off, the second transistor Q6 is turned off, such that the connection point of electrode 1 # is disconnected from the ground. A first resistor R5 achieves the function of current limiting and protection, such that the first transistor Q3 is ensured to operate safely and stably under control of a control signal from the controller.

Hereinafter, circuit structures of the current detection units are described.

The first current detection unit, the second current detection unit, the third current detection unit, and the fourth current detection unit all have the same circuit devices or elements. Using the first current detection unit as an example, the same structures and connections of the current detection units are described. As illustrated in FIG. 15A, the first current detection unit includes a third transistor Q4, a third resistor R6, a fourth resistor R7, and a diode D1. A collector of the third transistor Q4 is connected to the controller (connected to an MCU_CURRENT1 # of the controller), an emitter of the third transistor Q4 is grounded, a base of the third transistor Q4 is connected to both one terminal of the third resistor R6 and one terminal of the fourth resistor R7, the other terminal of the third resistor R6 is grounded, the other terminal of the fourth resistor R7 is connected to both the other terminal of the third resistor R6 and a positive terminal of the diode D1, and a negative terminal of the diode D1 is connected to the electrode connector (electrode 1 #) corresponding to the first current detection unit. The diode D1 is a Zener diode.

The current detection unit is configured to detect whether a current flows through an electrode connection point. For example, in a case where a current flows through the connection point of electrode 1 #, the third resistor R6 and the fourth resistor R7 form a voltage divider circuit, and a voltage at the connection point of electrode 1 # is divided and then fed to the base of the third transistor Q4. The third transistor Q4 is turned on, such that the current flows through the emitter and the collector of the third transistor Q4. The controller detects presence of the current based on an MCU_CURRENT1 # signal. The diode D1 is configured to protect the base of the third transistor Q4 from over-voltage damages.

Hereinafter, circuit structures of the current control units are described.

The first current control unit, the second current control unit, the third current control unit, and the fourth current control unit all have the same circuit devices or elements. Using the first current control unit as an example, the same structures and connections of the current control units are described. As illustrated in FIG. 15A, the first current detection unit includes a resistor R1, a transistor Q1, an inductor U1, a diode U2, and a capacitor C1. The capacitor C1 is a polarized capacitor. One terminal of the resistor R1 is connected to the controller (connected to the MCU_PWM1 pin of the controller), the other terminal of the resistor R1 is connected to a base of the transistor Q1, one terminal of the inductor U1 is connected to the power terminal VCC, the other terminal of the inductor U1 is connected to both a collector of the transistor Q1 and a positive terminal of the diode U2, a negative terminal of the diode U2 is connected to both a positive terminal of the capacitor C1 and the emitter of the second transistor Q5, and an emitter of the transistor Q1 and a negative terminal of the capacitor C1 are grounded.

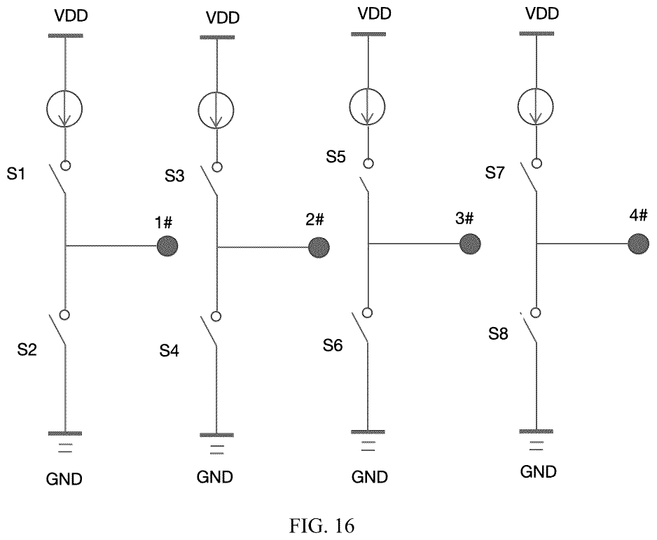

The current control unit is configured to control an intensity of the current output to the electrode connection point. The inductor U1 and the diode U2 form a boost or buck circuit, configured to stabilize the current. The transistor Q1 serves as a switching transistor, and is controlled to be turned on based on an MCU_PWM1 signal output by the controller, such that the output current is regulated. In a case where the transistor Q1 is turned on, the inductor U1 stores energy; or in a case where the transistor Q1 is turned off, the inductor U1 releases the stored energy, and power is supplied to the connection point of electrode 1 # via the diode U2. The alternate turn-on or turn-off of the transistor Q1 may achieve stable current output, and an ON time of the transistor Q1 determines a period within which the inductor U1 stores and releases energy. By adjusting a duty cycle of the MCU_PWM1 signal (a ratio of an ON time to a total period), a magnitude of the output current may be regulated. The capacitor C1 is configured to filter high-frequency noise and ensure stability of the output current, such that a smooth current is output. Hereinafter, a path detection process of a therapeutic device having the path detection circuit as illustrated in FIG. 15A to FIG. 15D is described. The circuit structure illustrated in FIG. 15A to FIG. 15D is simplified to obtain the circuit diagram in FIG. 16. In the related circuit of the first electrode connector, 1 # represents the first electrode connector, switch S1 is the first switch, switch S2 is the second switch, VDD represents the power terminal, GND is the ground, correspondence relationships of the related circuits of the second electrode connector to the fourth electrode connector are similar.

Referring to FIG. 16, upon power on of the main unit of the therapeutic device, the controller performs the following operations:

In S101, switch S1 is controlled to be turned on, switches S4, S6, and S8 are sequentially controlled to be turned on, and a current flowing through electrode 1 # is detected to detect whether the connection point of electrode 1 # forms paths with connection points of electrode 2 #, electrode 3 #, and electrode 4 # respectively.

In a case where switch S1 and switch S4 are turned on, in response to detecting a current, the connection points of electrode 1 # and electrode 2 # have formed conductive paths with the skin of the human body, the connection points may serve as two electrodes of a channel, and an electrical pulse stimulation signal may be transmitted to the connection points of electrode 1 # and electrode 2 #; and in response to detecting no current, the connection points of electrode 1 # and electrode 2 # have not formed conductive paths with the skin of the human body, and no electrical pulse stimulation signal may be transmitted to the connection points of electrode 1 # and electrode 2 #. By either detecting the current flowing through the connection point of electrode 1 # or detecting a current flowing through the connection point of electrode 2 #, whether the connection point of electrode 1 # forms a path with the connection point of electrode 2 # may be detected.

Subsequent to detection formation of the path between the connection point of electrode 1 # and the connection point of electrode 2 #, switch S1 is maintained on, switch S4 is turned off, and switch S6 is turned on. In a case where switch S1 and switch S6 are turned on, in response to detecting a current, the connection points of electrode 1 # and electrode 3 # have formed conductive paths with the skin of the human body, the connection points may serve as two electrodes of a channel, and an electrical pulse stimulation signal may be transmitted to the connection points of electrode 1 # and electrode 3 #; and in response to detecting no current, the connection points of electrode 1 # and electrode 3 # have not formed conductive paths with the skin of the human body, and no electrical pulse stimulation signal may be transmitted to the connection points of electrode 1 # and electrode 3 #. By either detecting the current flowing through the connection point of electrode 1 # or detecting a current flowing through the connection point of electrode 3 #, whether the connection point of electrode 1 # forms a path with the connection point of electrode 3 # may be detected.

Subsequent to detection of formation of the path between the connection point of electrode 1 # and the connection point of electrode 3 #, switch S1 is maintained on, switch S6 is turned off, and switch S8 is turned on. In a case where switch S1 and switch S8 are turned on, in response to detecting a current, the connection points of electrode 1 # and electrode 4 # have formed conductive paths with the skin of the human body, the connection points may serve as two electrodes of a channel, and an electrical pulse stimulation signal may be transmitted to the connection points of electrode 1 # and electrode 4 #; and in response to detecting no current, the connection points of electrode 1 # and electrode 4 # have not formed conductive paths with the skin of the human body, and no electrical pulse stimulation signal may be transmitted to the connection points of electrode 1 # and electrode 4 #. By either detecting the current flowing through the connection point of electrode 1 # or detecting a current flowing through the connection point of electrode 4 #, whether the connection point of electrode 1 # forms a path with the connection point of electrode 4 # may be detected.

In S102, switch S3 is controlled to be turned on, switches S6 and S8 are sequentially controlled to be turned on, and a current flowing through electrode 2 # is detected to detect whether the connection point of electrode 2 # forms paths with connection points of electrode 3 # and electrode 4 # respectively.

Subsequent to detection of formation of the paths between the connection point of electrode 1 # and the connection points of electrode 2 #, electrode 3 #, and electrode 4 #, switch S1 and switch S8 are turned off, and then switch S3 and switch S6 are controlled to be turned on.

In a case where switch S3 and switch S6 are turned on, in response to detecting a current, the connection points of electrode 2 # and electrode 3 # have formed conductive paths with the skin of the human body, the connection points may serve as two electrodes of a channel, and an electrical pulse stimulation signal may be transmitted to the connection points of electrode 2 # and electrode 3 #; and in response to detecting no current, the connection points of electrode 2 # and electrode 3 # have not formed conductive paths with the skin of the human body, and no electrical pulse stimulation signal may be transmitted to the connection points of electrode 2 # and electrode 3 #. By either detecting the current flowing through the connection point of electrode 2 # or detecting a current flowing through the connection point of electrode 3 #, whether the connection point of electrode 2 # forms a path with the connection point of electrode 3 # may be detected.

Subsequent to detection of formation of the path between the connection point of electrode 2 # and the connection point of electrode 3 #, switch S3 is maintained on, switch S6 is turned off, and switch S8 is turned on. In a case where switch S3 and switch S8 are turned on, in response to detecting a current, the connection points of electrode 2 # and electrode 4 # have formed conductive paths with the skin of the human body, the connection points may serve as two electrodes of a channel, and an electrical pulse stimulation signal may be transmitted to the connection points of electrode 2 # and electrode 4 #; and in response to detecting no current, the connection points of electrode 2 # and electrode 4 # have not formed conductive paths with the skin of the human body, and no electrical pulse stimulation signal may be transmitted to the connection points of electrode 2 # and electrode 4 #. By either detecting the current flowing through the connection point of electrode 2 # or detecting a current flowing through the connection point of electrode 4 #, whether the connection point of electrode 2 # forms a path with the connection point of electrode 4 # may be detected.

In S103, switch S5 is controlled to be turned on, switch S8 is turned on, and a current flowing through the connection point of electrode 3 # is detected.

Subsequent to detection of formation of the paths between the connection point of electrode 2 # and the connection points of electrode 3 # and electrode 4 #, switch S3 and switch S8 are turned off, and then switch S5 and switch S8 are controlled to be turned on.

In a case where switch S5 and switch S8 are turned on, in response to detecting a current, the connection points of electrode 3 # and electrode 4 # have formed conductive paths with the skin of the human body, the connection points may serve as two electrodes of a channel, and an electrical pulse stimulation signal may be transmitted to the connection points of electrode 3 # and electrode 4 #; and in response to detecting no current, the connection points of electrode 3 # and electrode 4 # have not formed conductive paths with the skin of the human body, and no electrical pulse stimulation signal may be transmitted to the connection points of electrode 3 # and electrode 4 #. By this step, a current flowing through the connection point of electrode 4 # may also be detected, to detect whether the connection point of electrode 3 # forms a path with the connection point of electrode 4 #.

By the above steps, subsequent to determination of two electrode connectors that form a path, an electrical pulse stimulation signal may be output to the path formed between these two electrode connectors.

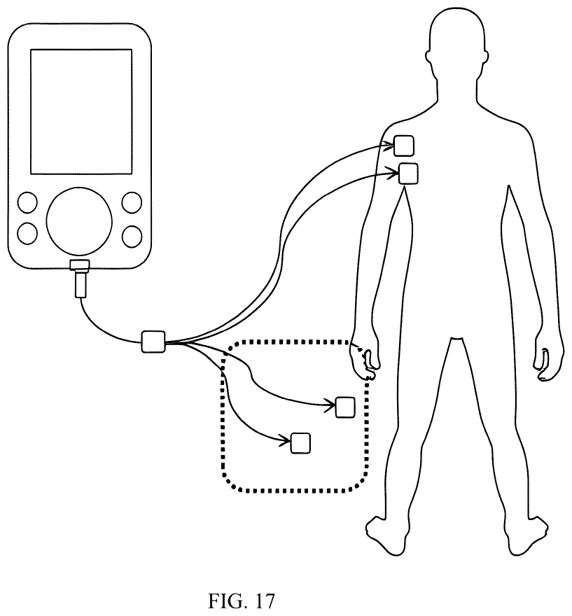

For example, as illustrated in FIG. 17, the electrode pads connected to the connection points of electrode 1 # and electrode 2 # are attached to the human body, and the electrode pads connected to the connection points of electrode 3 # and electrode 4 # are not attached to the human body. In response to determination that a path is formed between the connection points of electrode 1 # and electrode 2 #, two groups of switches (including switches S1 and S4, and switches S2 and S3) are alternately controlled to be turned on. For example, within a first time period, switch S1 and switch S4 are turned on and a positive pulse is output; and within a subsequent second time period, switch S2 and switch S3 are turned on and a negative pulse is output, such that positive and negative pulses are alternately output. Nevertheless, the first group of switches may be controlled to maintain constantly on, and a unidirectional pulse is output. The main unit of the therapeutic device outputs electrical pulses stimulation signals based on stimulation modes configured in the therapeutic device. The connectors of electrode 3 # and electrode 4 # in FIG. 17 that are not attached to the human body may be magnetically connected to each other.

It should be finally noted that the above-described embodiments are merely for illustration of the present disclosure, but are not intended to limit the present disclosure. Although the present disclosure is described in detail with reference to these embodiments, a person skilled in the art may also make various modifications to the technical solutions disclosed in the embodiments, or make equivalent replacements to a part of or all technical features contained therein. Such modifications or replacement, made without departing from the principles of the present disclosure, shall fall within the scope defined by the claims and the specification of the present disclosure. Especially, various technical features mentioned in various embodiments may be combined in any fashion as long as there is no structural conflict. The present disclosure is not limited to the specific embodiments described herein in this specification, but also includes all the technical solutions falling within the scope subject to the appended claims.

What is claimed is:

1. An electrode head for a therapeutic device, comprising:
at least one wire;
a main unit connector disposed at a first end of the at least one wire, and electrically connected to a main unit of the therapeutic device; and
at least one electrode connector disposed at a second end of the at least one wire, wherein the at least one electrode connector comprises an insulating enclosure and a conductive portion, the conductive portion being electrically connected to the main unit connector via the at least one wire, the conductive portion comprising a contact surface configured to mate with an electrode sheet, and the insulating enclosure covering the conductive portion with a contact surface exposed;
wherein a magnet is disposed within the conductive portion, the magnet being magnetized along an axial direction of the magnet, the axial direction of the magnet being perpendicular to the contact surface; and the insulating enclosure comprises a first end surface and a second end surface that are opposite along the axial direction of the magnet, the contact surface being exposed at the first end surface, the first region of the second end surface and the contact surface being both planar, the first region being magnetically attachable to the contact surface of another electrode connector, and a magnetic pole mounting direction of the magnet in each of the at least one electrode connector being consistent.

2. The electrode head for the therapeutic device according to claim 1, wherein a protrusion is arranged on the second end surface of the insulating enclosure, wherein a top surface of the protrusion forms the first region; and a recess is defined in the first end surface of the insulating enclosure, wherein a shape and size of the recess are configured to be adapted for engagement with a protrusion of another electrode connector.

3. The electrode head for the therapeutic device according to claim 2, wherein a cross-section of the protrusion and a cross-section of the recess are both circular, or square, or triangular, or elliptical.

4. The electrode head for the therapeutic device according to claim 2, wherein a bottom surface of the recess is flush with the contact surface, and a height of the protrusion is equal to a depth of the recess.

5. The electrode head for the therapeutic device according to claim 1, wherein the insulating enclosure comprises a first housing and a second housing, wherein a first engagement portion is arranged on the first housing, and a second engagement portion is arranged on the second housing, wherein the first engagement portion and the second engagement portion are engaged with each other to secure the first housing and the second housing.

6. The electrode head for the therapeutic device according to claim 5, wherein the first engagement portion is an arcuate recess, and the second engagement portion is an arcuate protrusion, wherein the arcuate recess is adapted to the arcuate protrusion in terms of shape and size.

7. The electrode head for the therapeutic device according to claim 5, wherein a through hole is defined in the first housing, wherein the through hole is configured to accommodate the conductive portion.

8. The electrode head for the therapeutic device according to claim 1, wherein the magnet is a ring-shaped body.

9. The electrode head for the therapeutic device according to claim 1, wherein a number of the at least one wire is n, and a number of the at least one electrode connector is n, wherein n is a positive integer greater than 1, and the n electrode connectors are electrically connected to the main body connector via the n wires; and
the electrode lead for the therapeutic device is configured such that: in a non-use state, the n electrode connectors are disposed adjacently, and in any adjacent two of the n electrode connectors, a contact surface of one electrode connector is magnetically attached to a first region of the other electrode connector such that the electrode lead for the therapeutic device is stored.

10. The electrode head for the therapeutic device according to claim 1, wherein the at least one wire comprises a first wire and a second wire, and the at least one electrode connector comprises a first electrode connector and a second electrode connector, wherein conductive portions of the first and second electrode connectors are electrically connected to the main unit connector via the first and second wires respectively; and
the electrode lead for the therapeutic device is configured such that: in a non-use state, a contact surface of the first electrode connector is magnetically attached to a first region of the second electrode connector such that the electrode lead for the therapeutic device is stored.

11. The electrode head for the therapeutic device according to claim 1, wherein the at least one wire comprises a first wire, a second wire, a third wire, and a fourth wire, and the at least one electrode connector comprises a first electrode connector, a second electrode connector, a third electrode connector, and a fourth electrode connector, wherein conductive portions of the first, second, third, and fourth electrode connectors are electrically connected to the main unit connector via the first, second, third, and fourth wires respectively; and
the electrode lead for the therapeutic device is configured such that: a contact surface of the first electrode connector is magnetically attached to a first region of the second electrode connector, and a contact surface of the third electrode connector is magnetically attached to a first region of the fourth electrode connector, such that a pair of electrode connectors for single-channel use is obtained; and
the electrode lead for the therapeutic device is further configured such that: in a non-use state, the contact surface of the first electrode connector is magnetically attached to the first region of the second electrode connector, a contact surface of the second electrode connector is magnetically attached to a first region of the third electrode connector, and the contact surface of the third electrode connector is magnetically attached to the first region of the fourth electrode connector, such that the electrode lead for the therapeutic device is stored.

12. A therapeutic device, comprising:
an electrode head; and
a main unit; wherein the electrode head comprises:
at least one wire;
a main unit connector disposed at a first end of the at least one wire, and electrically connected to a main unit of the therapeutic device; and
at least one electrode connector disposed at a second end of the at least one wire, wherein the at least one electrode connector comprises an insulating enclosure and a conductive portion, the conductive portion being electrically connected to the main unit connector via the at least one wire, the conductive portion comprising a contact surface configured to mate with an electrode sheet, and the insulating enclosure covering the conductive portion with a contact surface exposed;
wherein a magnet is disposed within the conductive portion, the magnet being magnetized along an axial direction of the magnet, the axial direction of the magnet being perpendicular to the contact surface; and the insulating enclosure comprises a first end surface and a second end surface that are opposite along the axial direction of the magnet, the contact surface being exposed at the first end surface, the first region of the second end surface and the contact surface being both planar, the first region being magnetically attachable to the contact surface of another electrode connector, and a magnetic pole mounting direction of the magnet in each of the at least one electrode connector being consistent.

13. The therapeutic device according to claim 12, wherein a number of the at least one wire is n, and a number of the at least one electrode connector is n, wherein n is a positive integer greater than 1; and the main unit comprises a controller, n switch units, and n current detection units, wherein the n switch units are in one-to-one correspondence with the n electrode connectors, the n current detection units are in one-to-one correspondence with the n electrode connectors, the n electrode connectors comprise a first electrode connector, the n switch units comprise a first switch unit in correspondence with the first electrode connector, and the n current detection units comprise a first current detection unit in correspondence with the first electrode connector, wherein the first switch unit is connected to a power terminal of the main unit, and the other switches in the n switch units are grounded;
wherein the controller is configured to control the first switch unit to be turned on and control a target switch unit in the other switch units to be turned on, and detect a current at the first electrode connector via the first current detection unit or detect a current at a target electrode connector corresponding to a target current detection unit in the n current detection units via the target current detection unit to determine whether a path is formed between the first electrode connector and the target electrode connector, wherein the target electrode connector is in correspondence with the target switch unit.

14. The therapeutic device according to claim 12, wherein the at least one wire comprises a first wire, a second wire, and a third wire; the at least one electrode lead comprises a first electrode lead, a second electrode lead, and a third electrode lead; and the main unit comprises a controller, a first switch unit, a fourth switch unit, a sixth switch unit, a first current detection unit, a second current detection unit, and a third current detection unit; wherein the first switch unit, the fourth switch unit, and the sixth switch unit are in one-to-one correspondence with the first electrode lead, the second electrode lead, and the third electrode lead; the first current detection unit, the second current detection unit, and the third current detection unit are in one-to-one correspondence with the first electrode lead, the second electrode lead, and the third electrode lead; the first switch unit is connected to a power terminal of the main unit; and the fourth switch unit and the fourth switch unit are grounded;
wherein the controller is configured to control the first switch unit to be turned on and control a target switch unit in the fourth switch unit and the sixth switch unit to be turned on, and detect a current at the first electrode connector via the first current detection unit or detect a current at a target electrode connector corresponding to a target current detection unit in the second current detection unit and the third current detection unit via the target current detection unit to determine whether a path is formed between the first electrode connector and the target electrode connector, wherein the target electrode connector is in correspondence with the target switch unit.

15. The therapeutic device according to claim 12, wherein the at least one wire comprises a first wire, a second wire, a third wire, and a fourth wire; the at least one electrode lead comprises a first electrode lead, a second electrode lead, a third electrode lead, and a fourth electrode lead; and the main unit comprises a controller, a first switch unit, a fourth switch unit, a sixth switch unit, an eighth switch unit, a first current detection unit, a second current detection unit, a third current detection unit, and a fourth current detection unit; wherein the first switch unit, the fourth switch unit, the sixth switch unit, and the eighth switch unit are in one-to-one correspondence with the first electrode lead, the second electrode lead, the third electrode lead, and the fourth electrode lead; the first current detection unit, the second current detection unit, the third current detection unit, and the fourth current detection are in one-to-one correspondence with the first electrode lead, the second electrode lead, the third electrode lead, and the fourth electrode lead; the first switch unit is connected to a power terminal of the main unit; and the fourth switch unit, the fourth switch unit, and the eighth switch unit are grounded;
wherein the controller is configured to control the first switch unit to be turned on and control a target switch unit in the fourth switch unit, the sixth switch unit, and the eight switch unit to be turned on, and detect a current at the first electrode connector via the first current detection unit or detect a current at a target electrode connector corresponding to a target current detection unit in the second current detection unit, the third current detection unit, and the fourth current detection unit via the target current detection unit to determine whether a path is formed between the first electrode connector and the target electrode connector, wherein the target electrode connector is in correspondence with the target switch unit.

16. The therapeutic device according to claim 12, wherein the at least one wire comprises a first wire, a second wire, a third wire, and a fourth wire; the at least one electrode lead comprises a first electrode lead, a second electrode lead, a third electrode lead, and a fourth electrode lead; and the main unit comprises a controller, a first switch, a third switch, a fourth switch, a fifth switch unit, a sixth switch,, an eighth switch, a first current detection unit, a second current detection unit, a third current detection unit, and a fourth current detection unit; wherein the first electrode connector is connected to a power terminal of the main unit via the first switch, the second electrode is connected to the power terminal of the main unit via the third switch and grounded via the fourth switch, the third electrode connector is connected to the power terminal of the main unit via the fifth switch and grounded via the sixth switch, the fourth electrode connector is grounded via the eighth switch, and the first current detection unit, the second current detection unit, the third current detection unit, and the fourth current detection unit are respectively connected to the first electrode connector, the second electrode connector, the third electrode connector, and the fourth electrode connector;
the controller is configured to:
control the first switch and the fourth switch to be turned on, and detect a current at the first electrode connector via the first current detection unit or detect a current at the second electrode connector via the second current detection circuit to determine whether a path is formed between the first electrode connector and the second electrode connector;
control the first switch and the sixth switch to be turned on, and detect a current at the first electrode connector via the first current detection unit or detect a current at the third electrode connector via the third current detection circuit to determine whether a path is formed between the first electrode connector and the third electrode connector;
control the first switch and the eighth switch to be turned on, and detect a current at the first electrode connector via the first current detection unit or detect a current at the fourth electrode connector via the fourth current detection circuit to determine whether a path is formed between the first electrode connector and the fourth electrode connector;
control the third switch and the sixth switch to be turned on, and detect a current at the second electrode connector via the second current detection unit or detect a current at the third electrode connector via the third current detection circuit to determine whether a path is formed between the second electrode connector and the third electrode connector;
control the third switch and the eighth switch to be turned on, and detect a current at the second electrode connector via the second current detection unit or detect a current at the fourth electrode connector via the fourth current detection circuit to determine whether a path is formed between the second electrode connector and the fourth electrode connector; or
control the fifth switch and the eighth switch to be turned on, and detect a current at the third electrode connector via the third current detection unit or detect a current at the fourth electrode connector via the fourth current detection circuit to determine whether a path is formed between the third electrode connector and the fourth electrode connector.

17. The therapeutic device according to claim 16, wherein a first current control unit is further connected between the first switch and the power terminal of the main unit; and in a case where the first electrode connector forms a first path, a second path, or a third path with the second electrode connector, the third electrode connector, or the fourth electrode connector, the controller is configured to control a magnitude of current of the first path, the second path, or the third path via the first current control unit;
a second current control unit is further connected between the third switch and the power terminal of the main unit; and in a case where the first electrode connector forms a fourth path or a fifth path with the third electrode connector or the fourth electrode connector, the controller is configured to control a magnitude of current of the fourth path or the fifth path via the second current control unit; and
a third current control unit is further connected between the fourth switch and the power terminal of the main unit; and in a case where the third electrode connector forms a sixth path with the fourth electrode connector, the controller is configured to control a magnitude of current of the sixth path via the third current control unit.

18. The therapeutic device according to claim 17, wherein each of the first switch, the third switch, the fourth switch, the fifth switch, the sixth switch, and the eight switch comprises a first resistor, a first transistor, a second resistor, and a second transistor; wherein
one terminal of the first resistor is connected to the controller, the other terminal of the resistor is connected to a base of the first transistor, a collector of the first transistor is connected to one terminal of the second resistor, an emitter of the first transistor is grounded, and the other terminal of the second resistor is connected to a base of the second transistor;
for the first switch, an emitter of the second transistor is connected to the power terminal of the main unit via the first current control unit, and a collector of the second transistor is connected to the first electrode connector;
for the third switch, the emitter of the second transistor is connected to the power terminal of the main unit via the second current control unit, and the collector of the second transistor is connected to the second electrode connector;
for the fourth switch, the emitter of the second transistor is connected to the second electrode connector, and the collector of the second transistor is grounded via the second current detection unit;
for the fifth switch, the emitter of the second transistor is connected to the power terminal of the main unit via the third current control unit, and the collector of the second transistor is connected to the third electrode connector;
for the sixth switch, the emitter of the second transistor is connected to the third electrode connector, and the collector of the second transistor is grounded via the third current detection unit; and
for the eighth switch, the emitter of the second transistor is connected to the fourth electrode connector, and the collector of the second transistor is grounded via the fourth current detection unit.

19. The therapeutic device according to claim 13, wherein each of the current detection units comprises a third transistor, a third resistor, a fourth resistor, and a diode;
wherein a collector of the third transistor is connected to the controller, an emitter of the third transistor is grounded, a base of the third transistor is connected to both one terminal of the third resistor and one terminal of the fourth resistor, the other terminal of the third resistor is grounded, the other terminal of the fourth resistor is connected to both the other terminal of the third resistor and a positive terminal of the diode, and a negative terminal of the diode is connected to the electrode connector corresponding to the current detection unit.

20. The therapeutic device according to claim 16, wherein the main unit further comprises a second switch and a seventh switch, wherein the first electrode connector is grounded via the second switch, the fourth electrode connector is connected to the power terminal of the main unit via the seventh switch; wherein
in a case where the first electrode connector forms a first path with the second electrode connector, the controller is configured to alternately control a first group of switches and a second group of switches to be turned on to supply a bidirectional pulse to the first path, wherein the first group of switches comprises the first switch and the fourth switch, and the second group of switches comprises the second switch and the third switch;
in a case where the first electrode connector forms a second path with the third electrode connector, the controller is configured to alternately control a third group of switches and a fourth group of switches to be turned on to supply a bidirectional pulse to the second path, wherein the third group of switches comprises the first switch and the sixth switch, and the fourth group of switches comprises the second switch and the fifth switch;
in a case where the first electrode connector forms a third path with the fourth electrode connector, the controller is configured to alternately control a fifth group of switches and a sixth group of switches to be turned on to supply a bidirectional pulse to the third path, wherein the fifth group of switches comprises the first switch and the eighth switch, and the sixth group of switches comprises the second switch and the seventh switch;
in a case where the second electrode connector forms a fourth path with the third electrode connector, the controller is configured to alternately control a seventh group of switches and an eighth group of switches to be turned on to supply a bidirectional pulse to the fourth path, wherein the seventh group of switches comprises the third switch and the sixth switch, and the eighth group of switches comprises the fourth switch and the fifth switch;
in a case where the second electrode connector forms a fifth path with the fourth electrode connector, the controller is configured to alternately control a ninth group of switches and a tenth group of switches to be turned on to supply a bidirectional pulse to the fifth path, wherein the ninth group of switches comprises the third switch and the eighth switch, and the fourth group of switches comprises the fourth switch and the seventh switch; and
in a case where the third electrode connector forms a sixth path with the fourth electrode connector, the controller is configured to alternately control an eleventh group of switches and a twelfth group of switches to be turned on to supply a bidirectional pulse to the sixth path, wherein the eleventh group of switches comprises the fifth switch and the eighth switch, and the twelfth group of switches comprises the sixth switch and the seventh switch.
